(19) United States
(12) Patent Application Publication
(10) Pub. No.: US 2025/0275936 A1
(43) Pub. Date: Sep. 4, 2025

(54) METHOD FOR TREATING CANCERS AND NEUROLOGICAL DISEASES USING QUERCETIN-CONTAINING COMPOSITIONS

(71) Applicant: Quercis Pharma AG, Zug (CH)

(72) Inventor: Thomas Christian LINES, Oberwil-Lieli (CH)

(21) Appl. No.: 18/862,507

(22) PCT Filed: May 5, 2023
(86) PCT No.: PCT/IB2023/054726

§ 371 Date: Nov. 1, 2024

Related U.S. Application Data
(60) Provisional application No. 63/364,298, filed on May 6, 2022.

(51) Int. Cl. A61K 31/352; A61K 31/375; A61K 31/404; A61K 31/4406; A61K 31/519; A61K 45/06; A61P 25/28

(52) U.S. Cl. CPC A61K 31/352; A61K 31/375; A61K 31/404; A61K 31/4406; A61K 31/519; A61K 45/06; A61P 25/28

(57) ABSTRACT

Compositions and methods for treating a cancer or a neurological disease. A method of treating a cancer or a neurological disease comprising administering to a subject in need thereof an effective amount of quercetin, vitamin B3, vitamin C, folic acid, a chemotherapeutic agent, and zafirlukast. A method of treating amyotrophic lateral sclerosis comprising administering to a subject in need thereof an effective amount of quercetin, vitamin B3, vitamin C, folic acid, a chemotherapeutic agent, and zafirlukast.

### CROSS-REFERENCE TO RELATED APPLICATIONS

This Application claims priority to U.S. Provisional Application No. 63/364,298 filed on May 6, 2022, which is incorporated herein by reference.

### SUMMARY

The present disclosure relates to a method of treating a cancer or a neurological disease, the method comprising administering to a subject in need thereof an effective amount of quercetin, vitamin B3, vitamin C, a chemotherapeutic agent, and zafirlukast, and optionally folic acid.

### BRIEF DESCRIPTION OF THE DRAWINGS

FIG. 1 shows a schematic diagram of the cells important in ALS pathology.

FIG. 2 shows an image of microglia in the spinal cord of SOD1G93A rats treated with IsoQ vs. Vehicle stained for the senescence marker p16.

FIG. 3 shows an image of cultured microglia treated with IsoQ and assessed for β-Galactosidase (β-Gal) activity.

FIG. 4 quantifies β-Gal activity in cultured microglia treated with vehicle or IsoQ.

FIG. 5A quantifies the concentration of cultured microglia that are β-Gal positive.

FIG. 5B is a representative scatterplot.

FIG. 5C is a representative quantification of β-Gal activity.

FIG. 5D is a representative scatterplot of DNA content.

FIG. 5E is a representative scatterplot of DNA content.

FIG. 6 shows images of misfolded SOD1 protein in spinal motor neurons after treatment with IsoQ or vehicle.

FIG. 7 shows images of damaged motor neurons stained for ubiquitin (top) or p75NTR (bottom) treated with IsoQ or vehicle.

FIG. 8 shows images of TDP-43 aggregates in motor neurons treated with IsoQ or vehicle.

FIG. 9 shows images of the ventral horn of the spinal cord in SODIG93A rats treated with IsoQ or vehicle and stained with the astrocyte marker glial fibrillary acidic protein (GFAP).

FIG. 10 shows images of the ventral horn of the spinal cord in SODIG93A rats treated with IsoQ or vehicle and stained with Iba1 to visualize microglia.

FIG. 11 quantifies the percent of SOD1G93A rats symptomatic for paralysis after treatment with vehicle or IsoQ.

FIG. 12 shows images of protein disulphide isomerase (PDI) expression in motor neurons treated with vehicle or IsoQ.

FIG. 13 shows images of myofibers from SODIG93A rat muscle after treatment with vehicle or IsoQ.

### DEFINITIONS

As used herein, “quercetin” refers to both quercetin aglycone and quercetin derivatives, e.g., quercetin-3-O-glucoside (also known as isoquercetin), quercetin-5-O-glucoside, quercetin-7-O-glucoside, quercetin-9-O-glucoside quercetin-3′-O-glucoside, quercetin-4′-O-glucoside, quercetin-3-O-rutinoside (also known as rutin), quercetin-3-O-[a-rhamnosyl-(1→2)-a-rhamnosyl-(1→6)]-13-glucoside, quercetin-3-O-galactoside, quercetin-7-O-galactoside, quercetin-3-O-rhamnoside, quercetin-7-O-galactoside, quercetin-glycoside, 7- hydroxyflavone, and any pharmaceutically acceptable salts thereof. “Quercetin” may also refer to isoquercetin or rutin or any constituent of rutin or isoquercetin, or metabolite or rutin or isoquercetin or quercetin, whether sulphated, glucuronidated or methylated form of rutin or quercetin, and any pharmaceutically acceptable salts thereof.

As used herein, “Vitamin B3” refers to vitamin B3 in its various forms, niacinamide, nicotinic acid, nicotinamide, and inositol hexanicotinate.

As used herein, “Vitamin C” refers to vitamin C (i.e., L-ascorbic acid, D-ascorbic acid, or both) and its salts (e.g., sodium ascorbate).

As used herein, “Folic acid” refers to a vitamin B complex that may also include vitamin B9, folate, pteroylglutamic acid, and L-methyl folate or 5-MTHF (5-methyltetrahydrofolate),

As used herein, “extracellular thiol isomerase” includes at least protein disulfide isomerase (PDI), thioredoxin (TRX), and the following endoplasmic reticulum resident proteins: ERp5, ERp57, and ERp72.

As used herein, “extracellular thiol isomerase inhibitor compound” is an inhibitor of one or more of the extracellular thiol isomerases. Exemplary extracellular thiol isomerase inhibitor compounds include zafirlukast, montelukast, CGP-13501 (CAS Reg. No. 56189-68-5), CGP-7930 (CAS Reg. No. 57717-80-3), alosetron, balsalazide, benserazide, butaclamol, leva-dopa, mesalazine, oxcarbazepine, a pharmaceutically acceptable salt, prodrug, and/or solid-state form thereof. As inhibitors of one or more of the extracellular thiol isomerases, one or more of these compounds can be used as an anti-thrombotic agent, an anticoagulant agent, an anti-inflammatory agent, an anti-viral agent, a chemotherapeutic or an anti-cancer agent, etc., or a combination thereof. Zafirlukast is a synthetic, selective peptide leukotriene receptor antagonist (LTRA), with the chemical name 4-(5-cyclopentyloxycarbonylamino-1-methylindol-3-ylmethyl)-3-methoxy-N-o-tolylsulfonylbenzamide. We have found zafirlukast to be a broad-spectrum thiol isomerase inhibitor that inhibits platelet function, thrombus formation and cancer cell growth. The synthesis and pharmaceutical forms of zafirlukast are further described in U.S. Pat. Nos. 4,859,692; 5,294,636; 5,319,097; 5,482,963; 5,583,152; 5,612,367; 6,143,775; 6,333,361; and 6,399,104, the contents of which are incorporated herein by reference in their entireties. Montelukast is a synthetic peptide leukotriene receptor antagonist (LTRA), with the chemical name [R-(E)]-1-[[[1-[3-[2-(7-chloro-2-quinolinyl)ethenyl]phenyl]-3-[2-(1-hydroxy-1-methylethyl) phenyl]propyl]thio]methylcyclo-propane acetic acid. The synthesis and pharmaceutical forms of montelukast and montelukast sodium are further described in U.S. Pat. No. 5,565,473, which is incorporated herein by reference in its entirety.

As used herein, the term “pharmaceutical agent” or “compound” refers to a chemical entity or biological product, or a combination of chemical entities or biological products, administered to a person to treat or prevent or control a disease or condition.

The term “active agent,” as used herein, means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound, crystalline forms, non-crystalline (i.e. amorphous) forms, and any polymorphs of the compound are included. All forms are contemplated herein regardless of the methods used to obtain them.

The term “chemotherapeutic agent,” as used herein, includes any compound or agent or composition that may directly or indirectly inhibit the uncontrolled growth and proliferation of cancer cells. Examples of chemotherapeutic agents include, but are not limited to, bevacizumab, irinotecan hydrochloride, cetuximab, oxaliplatin, leucovorin calcium, panitumumab, ziv-aflibercept, cyclophosphamide, everolimus, aldesleukin, quercetin aglycone, asparaginase, azacitidine, belzufitan, bendamustine, bleomycin, brentuximab vedotin, cabazitaxel, carmustine, chlorambucil, dactinomycin, eribulin mesylate, estramustine, hydroxyurea, ifosfamide, ixabepilone, mechlorethamine, melphalan, mitoxantrone, mitomycin, nelarabine, thioguanine, thiotepa, arsenic trioxide, decitabine, pegaspargase, procarbazine, romidepsin, streptozotocin, and vorinostat, alkylating agents such as altretamine, busulfan, carboplatin, cisplatin, cyclophosphamide, dacarbazine, lomustine, temozolomide, and trabectedin, antimetabolites such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine, and tipiracil, plant alkaloids such as vincristine, vinblastine, vinorelbine, paclitaxel, docetaxel, etoposide, teniposide, irinotecan, and topotecan, tyrosine kinase inhibitors such as everolimus, axitinib, sorafenib tosylate, pazopanib hydrochloride, sunitinib, temsirolimus, temozolomide, erlotinib, masitinib, naxitamab-gqgk, and regorafenib, and anti-tumor antibiotics such as daunorubicin, doxorubicin, doxorubicin hydrochloride, doxorubicin liposomal, epirubicin, idarubicin, and valrubicin, and combinations thereof. It will be obvious to one of ordinary skill in the art that other chemotherapeutic agents not listed here are within the scope of this disclosure.

The term “pharmaceutically acceptable salt,” as used herein, includes derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymandelic, phenylacetic, glutamic, benzoic, salicylic, mesylic, esylic, besylic, sulfanilic, 2-acetoxybenzoic, fumaric, toluenesulfonic, methanesulfonic, ethanedisulfonic, oxalic, isethionic, HOOC-(CH2)ₙ-COOH where n is 0-4, and the like. Lists of additional suitable salts may be found, e.g., in Remington's Pharmaceutical Sciences, 17th ed., Mack Publishing Company, Easton, Pa., p. 1418 (1985).

As used herein, the term “about” when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

The term “dosage form,” as used herein, means a unit of administration of an active agent. Examples of dosage forms include tablets, capsules, injections, suspensions, liquids, emulsions, creams, ointments, suppositories, inhalable forms, transdermal forms, and the like. An exemplary dosage form is a solid oral dosage form.

The term “pharmaceutical compositions,” as used herein, are compositions comprising at least one active agent or a pharmaceutically acceptable salt thereof, and at least one other substance, such as a carrier. Pharmaceutical compositions meet the U.S. FDA's GMP (good manufacturing practice) standards for human or non-human drugs. The pharmaceutical compositions can be formulated into a dosage form.

The term “carrier,” as used herein, applied to pharmaceutical compositions, refers to a diluent, excipient, or vehicle with which an active agent is provided. The term “carrier,” as used herein, applied to pharmaceutical compositions, refers to a diluent, excipient, or vehicle with which an active agent is provided. Classes of carriers include, for example, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional additional active agents may be included in a pharmaceutical composition, which do not substantially interfere with the activity of the active agent. The amount of carrier employed in conjunction with the compound is sufficient to provide a practical quantity of material for administration per unit dose of the active agent. Classes of carriers include, for example, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the active agent.

The term “patient” or “subject” as used herein, is a human or non-human animal in need of medical treatment. Medical treatment can include treatment of an existing condition, such as a disease or disorder, prophylactic or preventative treatment, or diagnostic treatment. In some embodiments the patient is a human patient.

As used herein, the terms “administer,” “administering” or “administration” as used herein refer to directly administering a compound or a composition to a subject.

The term “providing,” as used herein, means giving, administering, selling, distributing, transferring (for profit or not), manufacturing, compounding, or dispensing in each case optionally with or without a prescription.

The term “providing an extracellular thiol isomerase inhibitor compound or pharmaceutically acceptable salt thereof with at least one additional therapeutic agent,” as used herein, means an active agent or pharmaceutically acceptable salt thereof, and the additional active agent(s) are provided simultaneously in a single dosage form, provided concomitantly in separate dosage forms, or provided in separate dosage forms for administration separated by some amount of time that is within the time in which both the active agent or pharmaceutically acceptable salt thereof and the at least one additional active agent are within the bloodstream of a patient. The active agent or pharmaceutically acceptable salt thereof and the additional active agent need not be prescribed for a patient by the same medical care worker. The additional active agent or agents need not require a prescription. Administration of the active agent or pharmaceutically acceptable salt thereof or the at least one additional active agent can occur via any appropriate route, for example, oral tablets, oral capsules, oral liquids, inhalation, injection, suppositories or topical contact.

The term “treatment,” as used herein, includes providing an active agent or pharmaceutically acceptable salt thereof, either as the only active agent or together with at least one additional active agent sufficient to: (a) prevent a disease or condition or a symptom of a disease or condition from occurring in a patient who may be predisposed to the disease or condition but has not yet been diagnosed as having it; (b) inhibiting the disease or condition, i.e., arresting its development; and (c) relieving the disease or condition, i.e., causing regression of the disease or condition. As used herein, the terms “treat,” “treatment,” “treated,” or “treating” may also refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (that is, not worsening) of the state of the condition, disorder or disease; delay in onset or slowing of the progression of the condition, disorder or disease; amelioration of the condition, disorder or disease state; and remission or recurrence (whether partial or total), significantly reducing disease progression, whether or not it translates to immediate lessening of actual clinical symptoms, or enhancement or improvement of the condition, disorder or disease; preventing spread of the condition, disorder or disease state. Treatment seeks to elicit a clinically significant response without excessive levels of side effects. “Treating” and “treatment” also mean providing a therapeutically effective amount of an active agent or pharmaceutically acceptable salt thereof, as the only active agent or together with at least one additional active agent to a patient suffering from a disease or condition influenced by the activity of one or more active agents. “A disease or condition influenced by the activity of one or more active agents” means the one or more active agents is implicated in the disease or condition.

As used herein, the term “effective amount” refers to an amount that results in measurable inhibition of at least one symptom or parameter of a specific disorder or pathological process. As used herein, the term “therapeutically effective amount” of compositions of the application is an amount which confers a therapeutic effect on the treated subject at a reasonable benefit/risk ratio applicable to any medical treatment. The therapeutic effect may be objective (that is, measurable by some test or marker) or subjective (that is, subject gives an indication of or feels an effect, or physician observes a change).

The term “therapeutically effective amount” of an active agent, as used herein, means an amount effective, when administered to a patient, to provide a therapeutic benefit such as a prevention, inhibition, or an amelioration of symptoms, e.g., to prevent the activation of mast cells and prevent the formation of mast cell activated cytokines in a patient suffering from amyotrophic lateral sclerosis (ALS). A therapeutically effective amount may vary according to factors such as the health, age, and weight of the patient, and the ability of the compound to elicit a desired response in the patient. Dosage regimens may be adjusted to provide the optimum therapeutic response. A therapeutically effective amount is also one in which any toxic or detrimental effects (e.g., side effects) of the active agent are outweighed by the therapeutically beneficial effects.

The term “synergistic effect” as used herein, refers to an interaction or cooperation giving rise to a whole that is greater than the simple sum of its parts. As used herein, the effect of isoquercetin or quercetin and zafirlukast produces results that are greater than either quercetin or zafirlukast alone.

The term “preventing” may be taken to mean to prevent a specific disorder, disease or condition and/or prevent the reoccurrence of a specific disorder, disease or condition.

As used herein, the term “prognosis” means the probable course and outcome of a disease, especially of the chances of recovery.

The term “unit dosage form” refers to physically discrete units suitable as a unitary dosage for human subjects and other animals, each unit containing a predetermined quantity of active material calculated to produce the desired therapeutic effect, in association with a suitable pharmaceutical excipient.

The terms “improving,” “enhancing,” “treating,” and “lowering” refer to the administration of an effective amount of a composition of the invention to a subject, who needs to improve one or more of the above-mentioned conditions or who has one or more of the just-mentioned disorders, or a symptom or a predisposition of one of more of the disorders or conditions, with the purpose to improve one or more of these conditions, or to prevent, cure, alleviate, relieve, remedy, or ameliorate one or more of these disorders, or the symptoms or the predispositions of one or more of them. The term “administration” covers oral or parenteral delivery to a subject a composition of the invention in any suitable form, e.g., food product, beverage, tablet, capsule, suspension, and solution. The term “parenteral” refers to subcutaneous, intracutaneous, intravenous, intramuscular, intraarticular, intraarterial, intra-synovial, intrasternal, intranasal, intrathecal, intralesional, and intracranial injection, as well as various infusion techniques. An “effective amount” refers to a dose of the composition that is sufficient to provide a physical benefit (e.g., improving endurance) or a therapeutic benefit (e.g., lowering cholesterol or C-reactive protein levels, or reducing the risk of atherosclerosis or heart diseases). Both in vivo and in vitro studies can be conducted to determine optimal administration routes and doses.

The term “disease” as used herein is intended to be generally synonymous, and is used interchangeably with, the terms “disorder,” “dysfunction,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a medical condition or disorder described in the present disclosure. Such administration encompasses co-administration of these therapeutic agents in a substantially simultaneous manner, such as in a single capsule, or dosage presentation, having a fixed ratio of active ingredients, or in multiple, separate capsules for each active ingredient. In addition, such administration also encompasses use of each type of therapeutic agent in a sequential manner in the same patient, with delivery of the individual therapeutics separated by 1-24 hours, 1-7 days, or 1 or more weeks. In either case, the treatment regimen will provide beneficial effects of the drug combination in treating the conditions or disorders described herein.

The term “synergistic effect” means that when two or more drugs or pharmaceutical compositions are used in combination, the total effect of the combination is greater than the sum of the effects of each individual component alone.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims), are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

In some embodiments, a method of treating a cancer or a neurological disease is provided, the method comprising administering to a subject in need thereof an effective amount of quercetin, vitamin B3, vitamin C, and zafirlukast. In some embodiments, a method of treating a cancer or a neurological disease is provided, the method comprising administering to a subject in need thereof an effective amount of quercetin, vitamin B3, vitamin C, and zafirlukast, wherein the cancer or a neurological disease is treated. Some embodiments further comprise administering an effective amount of folic acid. In some embodiments, the effective amount of quercetin is between about 500 mg to about 3000 mg. In some embodiments, the effective amount of vitamin B3 is from about 20 μg to about 3 g. In some embodiments, the effective amount of vitamin C is from about 200 μg to about 3 g. In some embodiments, the effective amount of folic acid is from about 1000 μg to about 3000 μg. In some embodiments, the effective amount of zafirlukast is from about 10 mg to about 240 mg.

In some embodiments, the effective amount is sufficient to halt progression of one or more of the symptoms of a cancer. In some embodiments, the effective amount is sufficient to halt progression of one or more of the symptoms of a neurological disease. In some embodiments, the effective amount is sufficient to treat the cancer or a neurological disease.

In some embodiments, a method of treating a cancer or a neurological disease is provided, the method comprising administering to a subject in need thereof an effective amount of quercetin, vitamin B3, and vitamin C. In some embodiments, a method of treating a cancer or a neurological disease is provided, the method comprising administering to a subject in need thereof an effective amount of quercetin, vitamin B3, and vitamin C, wherein the cancer or neurological disease is treated. Some embodiments further comprise administering to a subject in need thereof an effective amount of folic acid. In some embodiments, the effective amount of quercetin is from about 500 mg to about 3000 mg. In some embodiments, the effective amount of quercetin is from about 800 mg to about 2000 mg.

In some embodiments, a method of treating a cancer or a neurological disease is provided, the method comprising administering to a subject in need thereof an effective amount of zafirlukast. In some embodiments, the effective amount of zafirlukast is from about 10 mg to about 240 mg. In some embodiments, the cancer or neurological disease is treated. In some embodiments, the effective amount of zafirlukast is from about 40 mg to about 160 mg.

In some embodiments, a method of treating a cancer or a neurological disease is provided, the method comprising administering to a subject in need thereof an effective amount of zafirlukast and a chemotherapeutic agent. In some embodiments, the effective amount of zafirlukast is from about 10 mg to about 240 mg. In some embodiments, the effective amount of zafirlukast is from about 40 mg to about 160 mg. In some embodiments, the cancer or neurological disease is treated.

In some embodiments, a method of treating a cancer or a neurological disease is provided, the method comprising administering to a subject in need thereof an effective amount of quercetin, vitamin B3, vitamin C, zafirlukast, and a chemotherapeutic agent. In some embodiments, the cancer or neurological disease is treated. Some embodiments further comprise administering to a subject in need thereof an effective amount of folic acid. In some embodiments, the effective amount of quercetin is between about 500 mg to about 3000 mg. In some embodiments, the effective amount of vitamin B3 is from about 20 μg to about 3 g. In some embodiments, the effective amount of vitamin C is from about 200 μg to about 3 g. In some embodiments, the effective amount of folic acid is from about 1000 μg to about 3000 μg. In some embodiments, the effective amount of zafirlukast is from about 10 mg to about 240 mg.

### DETAILED DESCRIPTION

Various methods are described herein for the treatment of a cancer or a neurological disease. The methods include administration of at least one pharmaceutical composition to the subject. The treatment can reduce or eliminate the symptoms of the cancer or neurological disease. The treatment can halt further progression of the symptoms of the cancer or neurological disease. The treatment can slow further progression of the symptoms of the cancer or neurological disease.

In one embodiment, the present invention describes a method of treating a cancer or a neurological disease, the method comprising administering to a subject in need thereof an effective amount of quercetin, vitamin B3, vitamin C, and zafirlukast. In some embodiments, the cancer or neurological disease is treated. Some embodiments further comprise administering to a subject in need thereof an effective amount of folic acid. The composition of this invention can be in various forms. In some embodiments, quercetin, vitamin B3, and vitamin C are administered in a single formulation, and zafirlukast is administered separately. In some embodiments, quercetin, vitamin B3, vitamin C, folic acid, and zafirlukast are administered separately. In some embodiments, quercetin, vitamin B3,vitamin C, and zafirlukast are administered in a single formulation. In some embodiments, quercetin, vitamin B3, vitamin C, and zafirlukast may be administered in one or more formulations, wherein any one of quercetin, vitamin B3, vitamin C, and zafirlukast may be administered in combination or alone. In some embodiments, quercetin, vitamin B3, and vitamin C are administered in a single formulation and folic acid and zafirlukast are administered separately. In some embodiments, quercetin, vitamin B3, vitamin C, folic acid, and zafirlukast are administered separately. In some embodiments, quercetin, vitamin B3, vitamin C, folic acid, and zafirlukast are administered in a single formulation. In some embodiments, quercetin, vitamin B3, vitamin C, folic acid, and zafirlukast may be administered in one or more formulations, wherein any one of quercetin, vitamin B3, vitamin C, folic acid, and zafirlukast may be administered in combination or alone. In some embodiments, the cancer or neurological condition is treated.

This invention is based, at least in part, on the unexpected findings that a composition comprising quercetin, vitamin B3, vitamin C, zafirlukast and optionally, folic acid, as active ingredients exhibits synergistic effects in treating patients with a cancer or neurological disease compared to treatment with either isoquercetin or quercetin or zafirlukast alone. Zafirlukast, a broad-spectrum thiol isomerase inhibitor, and isoquercetin or quercetin act largely on different thiol isomerases. Thiol isomerases, in general, can damage the spinal cord through mast cell activation, aberrant mast cell activity, and mitochondrial dysfunction, which lead to neuroinflammation, microglia activation and damage, and cell death. At low doses that are well tolerated by humans and animals, zafirlukast protects motor neurons against neuroinflammation, and quercetin and isoquercetin are known to protect against mitochondrial dysfunction and preserves mitochondrial biogenesis. Moreover, the inventors have observed the surprising result that these compounds, at low doses, act synergistically to efficiently protect motor units. The composition comprising quercetin, vitamin B3, vitamin C, and zafirlukast represents a surprising and substantial improvement in the treatment of cancer or neurological disease subjects. In some embodiments, the composition further comprises folic acid.

In some embodiments, the neurological disease is selected from age-associated memory impairment (AAMI), age-related cognitive decline (ARCD), agitation synucleinopathies, Alzheimer's disease (AD), Amyotrophic lateral sclerosis (ALS) dementia, autosomal-dominant Parkinson's disease, chemotherapy-induced neuropathy (CIPN), cognitive impairment no dementia (CIND), dementia, Creutzfeldt-Jakob disease (CJD), diffuse Lewy body disease (DLBD) also known as dementia with Lewy bodies (DLB), disorders or conditions characterized by the presence of Lewy bodies, Down syndrome, cerebral palsy, dyskinesia, epilepsy, frontotemporal dementia (FTD), HIV-associated neurocognitive disorder (HAND), HIV dementia, Huntington's disease, incidental LBD, inherited LBD, Lewy body dysphagia, mild cognitive impairment (MCI), multiple sclerosis, multiple system atrophy (MSA), neuropathies (including but not limited to peripheral neuropathy, diabetic neuropathy and retinal neuropathy), olivopontocerebellar atrophy, Parkinson's disease (PD), preclinical Alzheimer's disease (PCAD), psychiatric disorders (including but not limited to schizophrenia, bipolar disorders, depression, mania, anxiety disorders, post-traumatic stress disorders, delirium, eating disorders, autism, REM sleep behavior disorder, hallucinations, attention-deficit hyperactivity disorders, and psychosis), pure autonomic failure, seizures, Shy-Drager syndrome, striatonigral degeneration, synucleinopathies, traumatic brain injury (TBI), combined Alzheimer's and Parkinson disease and/or MSA, vascular dementia, diseases, disorders or conditions associated with abnormal expression, stability, activities and/or cellular processing of a-synuclein, diseases, disorders or conditions characterized by the presence of Lewy bodies, fibromyalgia, and combinations thereof.

In some embodiments, the cancer includes, but is not limited to, solid and liquid tumors, as well as to metastases of each. In some embodiments, the cancer is selected from ovarian cancer, lung cancer, prostate cancer, pancreatic cancer, colorectal cancer, endometrial cancer, brain/CNS cancer, liver cancer, thyroid cancer, bladder cancer, breast cancer, kidney cancer, cervical cancer, renal cancer, lymphoma, leukemia, skin cancer, urothelial cancer, glioblastoma, colorectal cancer, acute lymphocytic leukemia, acute myeloid leukemia, adrenal cancer, adult soft tissue sarcoma, anal cancer, aplastic anemia, basal and squamous cell skin cancer, bile duct cancer, bladder cancer, bone cancer, Castleman's disease, chronic lymphocytic leukemia, chronic myeloid leukemia, chronic myelomonocytic leukemia, endometrial cancer, esophageal cancer, Ewing family of tumors, eye cancer, gallbladder cancer, gastric cancer, gastrointestinal carcinoid, gastrointestinal stromal tumors, gestational trophoblastic disease, Hodgkin's disease, Kaposi's sarcoma, laryngeal and hypopharyngeal cancer, pediatric leukemia, non-small cell lung cancer, small cell lung cancer, lung carcinoid tumor, malignant mesothelioma, mastocystosis, melanoma skin cancer, multiple myeloma, myelodysplastic syndrome, nasal cavity and paranasal sinus cancer, nasopharyngeal cancer, neuroblastoma, non-Hodgkin's lymphoma, oral cavity and oropharyngeal cancer, osteosarcoma, penile cancer, pituitary tumors, renal cell carcinoma, retinoblastoma, rhabdomyosarcoma, salivary gland cancer, skin lymphoma, small intestine cancer, stomach cancer, testicular cancer, thymus cancer, thyroid cancer, uterine sarcoma, urothelial cancer, vaginal cancer, vulvar cancer, Waldenstrom's macroglobulinemia, and Wilms' tumor and combinations thereof.

### Pharmaceutical Compositions

Some embodiments are directed to pharmaceutical compositions for use in the methods disclosed herein comprising an effective amount of quercetin, vitamin B3, vitamin C, and a thiol isomerase inhibitor, wherein the thiol isomerase inhibitor is zafirlukast, montelukast, CGP-13501, CGP-7930, alosetron, balsalazide, benserazide, butaclamol, leva-dopa, mesalazine, oxcarbazepine, a pharmaceutically acceptable salt, prodrug, and/or a solid state form thereof. In some embodiments, the composition further comprises folic acid. In some embodiments, a method of treating a cancer or neurological disease comprises administering to a subject in need thereof an effective amount of quercetin, vitamin B3, vitamin C, and zafirlukast. In some embodiments, the method of treating cancer further comprises administering an effective amount of folic acid.

Some embodiments are directed to pharmaceutical compositions for use in the methods disclosed herein comprising an effective amount of quercetin, vitamin B3, vitamin C, and zafirlukast, wherein the composition includes about 500 mg to about 3000 mg of quercetin. In some embodiments, the composition includes about 800 mg to about 2000 mg of isoquercetin. In some embodiments, the composition further comprises folic acid. In some embodiments, the composition includes about 20 μg to about 3 g of vitamin B3. In some embodiments, the composition includes about 200 μg to about 3 g of vitamin C. In some embodiments, the composition includes about 1000 μg to about 3000 μg of folic acid. In some embodiments, the composition includes about 10 mg to about 240 mg of zafirlukast. In some embodiments, the composition includes about 10 mg to about 80 mg of zafirlukast. In some embodiments, the composition includes about 80 mg to about 160 mg of zafirlukast.

The weight ratio between quercetin, vitamin B3, vitamin C, in a composition of the invention can be 1:0.02-1:0.2-2.5, or any ratio in between. For example, the weight ratio can be 1:0.04-0.5:0.3-2.0, 1:0.05-0.3:0.4-1.5, 1:0.05-0.2:0.5-1, and 1:0.1-0.2:0.5-1. Preferred ratios include about 1:0.02:1, about 1:0.04:1, about 1:0.08:1, about 1:0.05:1.5, and about 1:0.16:1. Typically, a composition can be administered to a subject, once or periodically per day, with the composition in an amount that provides 500 mg to 3 g (preferably, 800 mg to 2.2 g) of quercetin.

After digestion, quercetin derivatives are converted to quercetin aglycone and other active derivatives, which are absorbed in the body. The quantity of quercetin mentioned above refers to that of quercetin aglycone or the quercetin moiety of a quercetin derivative. Quercetin can be added to a composition either in a pure form or as an ingredient in a mixture (e.g., a plant extract). Examples of commercially available quercetin include QU995 (containing 99.5% quercetin) and QU985 (containing 98.5% quercetin) from Quercegen Pharmaceuticals LLC (Boston, Mass.) and Merck KGaA (Brazil). Examples of commercially available isoquercetin include ISQ950AN (greater than or equal to 95% isoquercetin) and ISQ995AN (containing 99.5% isoquercetin) from Quercis Pharma AG (Zug, Switzerland).

Pharmaceutical compositions of this invention can be in various forms. For example, a composition may be a soft chew composition that includes quercetin, niacinamide, ascorbic acid, sodium ascorbate, sugar, corn syrup, sucralose, soy lecithin, corn starch, glycerin, palm oil, xylitol, carrageenan, FD&C Yellow #6, FD&C Yellow #5, and natural and/or artificial flavors. An exemplary serving of this soft chew composition may include 500 mg to 3000 mg of quercetin, along with vitamin B3 (i.e., niacinamide) and vitamin C (i.e., L-ascorbic acid and sodium ascorbate). Folic acid may be provided in the soft chew or separately in an amount of about 1000 μg to about 3000 μg. Zafirlukast may be provided in the soft chew or separately in an amount of about 10 mg to about 240 mg. A subject can take one to eight servings (e.g., 4 servings) of this soft chew composition daily. The amounts taken can vary depending on, for example, the disorder or condition to be treated and the physical states of the subject. Another exemplary composition of this soft chew includes 5.25 wt % of quercetin, 0.25 wt % of vitamin B3, and 7.81 wt % of vitamin C (i.e., L-ascorbic acid and sodium ascorbate).

Pharmaceutical compositions of this invention can further contain one or more active ingredients, such as an isoflavone (e.g., genistein or genistin), curcumin, resveratrol, isoquercetin, zafirlukast, luteolin, epigallocatechin gallate (EGCG), CoQ10, eicosapentaenoic acid (EPA), and docosahexaenoic acid (DHA). These active ingredients can be added to the composition either in a pure form or as a component in a mixture (e.g., an extract from a plant or an animal). A suitable daily dosage of each of these ingredients can vary depending on, for example, the disorder or condition to be treated and the physical states of the subjects. Exemplary daily dosages of some of these ingredients are: 20-2,500 mg (preferably 250-1,000mg) of curcumin, 10-1,000 mg (preferably 100-500 mg) of resveratrol, 500-3,000 mg (preferably 800-2000 mg) of isoquercetin, 10 mg to 240 mg of zafirlukast, 50-1,000 mg (preferably 100-700 mg) of EGCG, 25-300 mg (preferably 50-100 mg) of genistin/genistein, 10-1,000 mg (preferably 100-200 mg) of luteolin, 50-1,000 mg (preferably 70-500 mg) of EPA, and 50-1,000 mg (preferably 80-700 mg) of DHA. Further, it can be sweetened, if necessary, by adding a sweetener such as sorbitol, maltitol, hydrogenated glucose syrup and hydrogenated starch hydrolysate, high fructose corn syrup, cane sugar, beet sugar, pectin, or sucralose. The composition can also contain amino acids, fatty acids, proteins, fibers, minerals, a flavor enhancer, or a coloring agent. Exemplary amino acids include glycine, alanine, valine, leucine, isoleucine, proline, serine, threonine, cysteine, methionine, phenylalanine, tyrosine, tryptophan, aspartate, glutamate, histidine, lysine, arginine and their L-and D-configurations. Amino acids may be added to aid in digestion. Exemplary fatty acids include omega-3 fatty acids (e.g., linolenic acid), omega-6 fatty acids (e.g., linoleic acid), omega-9 fatty acids (e.g., oleic acid), sunflower oil, sunflower lecithin, soy oil, and soy lecithin. Exemplary proteins include plant proteins, such as soy proteins and chia seed proteins. Exemplary fibers include plant fibers, such as soy fibers and chia seed fibers. These ingredients can be added in the above-described composition either in a pure form or as a component in a mixture (e.g., an extract from a plant or an animal).

In some embodiments, pharmaceutical compositions can further comprise one or more exemplary fillers. Examples of exemplary fillers include cellulose and cellulose derivatives such as microcrystalline cellulose; starches such as dry starch, hydrolyzed starch, and starch derivatives such as corn starch; cyclodextrin; sugars such as powdered sugar, and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some embodiments, pharmaceutical compositions further comprise one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof. Disintegrants may be present in the composition from about 1 wt % to about 10 wt %, about 1wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, pharmaceutical compositions further comprise one or more binders, including but not limited to celluloses such as hydroxypropyl cellulose, methyl cellulose, and hydroxypropyl methylcellulose; starches such as corn starch, pregelatinized starch, and hydroxypropyl starch; waxes and natural and synthetic gums such as acacia, tragacanth, sodium alginate; synthetic polymers such as polymethacrylates and polyvinylpyrrolidone; and povidone, dextrin, pullulan, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some embodiments, pharmaceutical compositions further comprise one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1wt % to about 4 wt %, or about 0.1 wt % to about 5 wt % of the total weight of the composition, or any value between these ranges.

In some embodiments, pharmaceutical compositions further comprise one or more lubricants, including but not limited to stearic acid, magnesium stearate, calcium hydroxide, talc, corn starch, sodium stearyl fumarate, alkali metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, the pharmaceutical composition further comprises one or more glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, pharmaceutical compositions can be a tablet and further comprise a top coat, such as a hydroxypropyl-methylcellulose coating or a polyvinyl alcohol coating, and are available under the trade name Opadry, such as Opadry White and Opadry II (Opadry is a registered trademark of BPSI Holdings LLC, Wilmington, DE, USA). Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, pharmaceutical compositions can further comprise one or more preservative agents. Examples of preservative agents include sodium benzoate, paraoxybenzoic acid esters, methyl, ethyl, butyl, and propyl parabens, chlorobutanol, benzyl alcohol, phenylethyl alcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, pharmaceutical compositions can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

The pharmaceutical compositions can generally be in any physical form suitable for use in treating a subject. These forms can be referred to as a unit dosage form, such as an individual pill or tablet. In some embodiments, the pharmaceutical compositions can be formulated as tablets, capsules, granules, powders, liquids, suspensions, gels, syrups, slurries, suppositories, patches, nasal sprays, aerosols, injectables, implantable sustained-release formulations, or mucoadherent films. In some embodiments, the pharmaceutical compositions may be formed as a tablet, a bi-layer tablet, a capsule, a multiparticulate, a drug coated sphere, a matrix tablet, or a multicore tablet. A physical form can be selected according to the desired method of treatment.

The pharmaceutical compositions disclosed herein can be manufactured by various conventional methods such as conventional mixing, dissolving, granulating, dragee-making, levigating, emulsifying, encapsulating, entrapping, or lyophilizing processes. Pharmaceutical compositions can be formulated in a conventional manner using one or more physiologically acceptable carriers, diluents, excipients or auxiliaries that facilitate processing of the active agent into preparations that can be used pharmaceutically. Proper formulation can be selected upon the route of administration chosen.

When the above-described composition is in powder form, it can be used conveniently to prepare beverage, paste, jelly, capsules, or tablets. Lactose and corn starch are commonly used as diluents for capsules and as carriers for tablets. Lubricating agents, such as magnesium stearate, are typically included in tablets.

The composition of this invention can be a dietary supplement or a pharmaceutical formulation. As a dietary supplement, additional nutrients, such as minerals or amino acids may be included. The composition can also be a food product. As used herein, the term “food” broadly refers to any kinds of liquid and solid/semi-solid materials that are used for nourishing humans and animals, for sustaining normal or accelerated growth, or for maintaining stamina or alertness. Examples of human food products include, but are not limited to, tea-based beverages, juice, coffee, milk, jelly, cookies, cereals, chocolates, snack bars, herbal extracts, dairy products (e.g., ice cream, and yogurt), soybean products (e.g., tofu), and rice products.

### Method of Treating Cancer or Neurological Disease

In some embodiments, a method of treating a cancer or neurological disease, wherein the method comprises administering to a patient in need thereof a therapeutically effective amount of quercetin, vitamin B3, vitamin C, and zafirlukast. In some embodiments, a method of treating a cancer or neurological disease, wherein the method further comprises administering folic acid. In some embodiments, a method of treating a cancer or neurological disease, wherein the method comprises administering to a patient in need thereof a therapeutically effective amount of quercetin, vitamin B3, vitamin C, a thiol isomerase inhibitor, wherein the extracellular thiol isomerase inhibitor is zafirlukast, montelukast, CGP-13501, CGP-7930, alosetron, balsalazide, benserazide, butaclamol, leva-dopa, mesalazine, oxcarbazepine, a pharmaceutically acceptable salt, prodrug, and/or a solid state form thereof. In some embodiments, a method of treating a cancer or neurological disease, wherein the method further comprises administering folic acid.

Use of the described methods and pharmaceutical compositions can result in stopping, delaying, or reducing the progression of the disease, symptom, or other undesired property in a subject relative to a control population. The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, virus concentration, or other undesired property).

In some embodiments, a method of treating a cancer or neurological disease, wherein the method comprises administering to a patient or subject in need thereof a therapeutically effective amount of quercetin, vitamin B3, vitamin C, and zafirlukast, wherein the method of treating results in reduction of the symptoms of the cancer or neurological disease. In some embodiments, the method of treating a cancer or neurological disease further comprises administering an effective amount of folic acid.

The method of this invention significantly lowers both cholesterol and C-reactive protein levels, and increases the hemoglobin levels, the number of reticulocytes, and the mass of red blood cells. For example, it lowers the C-reactive protein levels in both subjects having higher than normal C-reactive protein levels and subjects having normal C-reactive protein levels. Further, this composition results in increased mitochondrial biogenesis or retention (e.g., in muscle and brain cells), reduced mitochondrial DNA damage and loss of mitochondria, or increased cytochrome C levels and citrate synthase activities. Thus, it is useful in treating disease associated with mitochondrial deficiencies in both humans and animals. This composition can also induce gene expression and activity of T-helper lymphocyte (Th-1) cytokines (e.g., interferon gamma) and can down-regulate T-helper lymphocyte 2 (Th-2) cytokines (e.g., interleukin 13). In addition, it can inhibit the expression and/or activity of one or more of the following three enzymes: matrix metalloproteinase 1 (MMP1), matrix metalloproteinase 2 (MMP2), and cyclooxygenase 2 (COX2). It can also block pathways mediated by epidermal growth factor receptor, such as epiregulin-mediated pathways. This composition can be used to prevent neurodegeneration by chelating excess iron accumulation in neurons. It also can be an activator of sirtuin and increase mitochondrial size, efficiency and production with SIRT 1, 2, 3, 4, PGC1 alpha, citrate synthase and cytochrome C all showing increased expression.

This method can also be used for treating diseases or disorders, such as a disorder associated with C-reactive protein, neurodegenerative disease (e.g., dyslexia, dyspraxia, autism, Asperger's disease, Alzheimer's disease, and mild cognitive impairment). “A disorder associated with C-reactive protein” refers to any disorder that results in an increase in the number of C-reactive proteins in blood, such as inflammation or a cardiovascular disorder (e.g., atherosclerosis, coronary heart disease, stroke, and peripheral arterial disease).

Further, this method can be used to treat certain symptoms of the above-mentioned diseases or disorders. For example, it can be used to lessen certain symptoms of a cancer or neurological disease, including muscle weakness, specifically weakness in legs, feet, or ankles, difficulty walking or performing daily activities, wasting of muscles, pain (such as facial pain or pain without apparent cause), muscle cramps, twitching in arms, shoulders, or tongue, electrical shock sensation, tumor growth, eating difficulty, fatigue, headaches, vision changes, hearing changes, swelling or lumps, skin changes, loss of awareness of location of body parts, loss of coordination (such as in speech), slurred speech, trouble swallowing, increased frequency of falls, loss of posture, shaking when performing fine movements, loss of ability to produce rapidly alternating movement (e.g., movement in a rhythm), and short-term or long term memory loss. This method can be used to improve lung function and chest expansion in patients. In addition, this method can also be used as a dietary supplement to improve the quality of life of a patient.

The method of this invention can be used to modulate cellular pathologies that underly cancer or neurological disease. For example, it can downregulate neurotoxic microglia and senescent microglia, reduce astrocytosis and microgliosis, improve spinal motor neuron pathology, prevent ubiquitin and p75NTR expression in damaged motor neurons, prevent motor neuron TDP-43 (TAR DNA-binding protein 43) aggregation, improve misfolded SOD1 (superoxide dismutase type 1).

The method of this invention can be used to reduce levels of neurofilament light chain in cerebral spinal fluid or blood serum in patients at risk for or symptomatic for cancer or neurological disease. The reduction in levels of neurofilament light chain using any formulation disclosed herein may be seen after administration for about 2 weeks to about 6 months or any amount of time within that range. The reduction in levels of neurofilament light chain may be systemic throughout the body. The reduction in levels of neurofilament light chain may be localized to the central nervous system, the peripheral nervous system, or the neuromuscular system.

The method of this invention can be used to target mitochondria pathology in motor axons and myofibers. For example, it can preserve mitochondrial number, mitochondrial integrity and size, and increase “long mitochondria domains” that are characteristic of healthy oxidative myofibers.

The method of this invention can act as a ferroportin inhibitor. For example, it can decrease serum iron, decrease ferritin, and decrease transferrin saturation coefficient (TSC).

The method of this invention can block extracellular protein disulfide isomerase (PDI) expression and associated inflammatory and neurotoxic pathways. For example, it can prevent PDI upregulation in surrounding motor neurons and prevent PDI initiated mast cell expression and infiltration.

The method described above can be preliminarily screened for efficacy in treating the above-described conditions by in vitro assays and then confirmed by animal experiments and clinic trials. Other suitable analytical and biological assays are apparent to those of ordinary skill in the art. For example, the bioavailability of quercetin can be measured by conducting pharmacokinetic studies and evaluated by the area under the curve in a plasma-drug concentration time curve.

The compounds and pharmaceutical compositions described herein may be administered at therapeutically effective dosage levels to treat the recited conditions, disorders, and diseases.

The compounds and pharmaceutical compositions described herein may be administered at prophylactically effective dosage levels to mitigate or prevent the recited conditions, disorders, and diseases.

The administration can be conducted daily for several days (for example about 2 to about 7 days), for several weeks (for example about 1 to about 4 weeks, specifically about 2 or about 3 weeks), or for several months (for example about 1 to about 36 months, specifically about 2 to about 24 months, and yet more specifically about 6 to about 12 months). In some embodiments, administration can be conducted from the time of diagnosis until the time wherein the disease or condition is deemed to be treated, or until death of the patient.

A therapeutically effective amount may range from about 0.001 pg/kg/day to about 500 mg/kg/day, preferably 0.01 pg/kg/day and 100 mg/kg/day. The skilled artisan will appreciate that certain factors may influence the dosage required to effectively treat a patient, including but not limited to the severity of the disease or disorder, previous treatments, the general health and/or age of the patient, and other diseases present. Moreover, treatment of a patient with a therapeutically effective amount of the compound can include a single treatment or can include a series of treatments. It will also be appreciated that the effective dosage of a compound used for treatment may increase or decrease over the course of a particular treatment.

The method of administering an effective amount of quercetin, vitamin B3, vitamin C, zafirlukast and optionally, folic acid, can occur once, twice, or three times a day to the patient in need thereof. Within this embodiment, the administration can be made orally.

Administration may be performed by generally any method. Methods of administering include infusion or injection of the composition. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some embodiments, the administering comprises oral delivery.

For injection, pharmaceutical compositions can be formulated in aqueous solutions, preferably in physiologically compatible buffers such as Hanks solution, Ringer's solution, or physiological saline buffer and/or in certain emulsion formulations. Solutions can contain one or more formulatory agents such as suspending, stabilizing and/or dispersing agents. In certain examples the pharmaceutical compositions can be provided in powder form for constitution with a suitable vehicle, for example, sterile pyrogen-free water, before use. For transmucosal administration, one or more penetrants appropriate to the barrier to be permeated can be used in the formulation. Such penetrants are generally known in the art.

For oral administration the composition may be formulated as tablets, pills, dragees, capsules, liquids, gels, syrups, slurries, suspensions, soft chews, and the like, for oral ingestion by a patient to be treated. For oral solid formulations such as, for example, the compositions may take the form of powders, capsules and tablets. For buccal administration, the compositions may take the form of tablets, lozenges, etc. formulated in conventional manner.

For administration by inhalation, the pharmaceutical composition can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, for example, gelatin, for use in an inhaler or insufflator, may be formulated containing a powder mix of the compound and a suitable powder base such as lactose or starch.

The subject can generally be any mammal. Examples of subjects include a primate, a human, a dog, a cat, a mouse, a rat, a cow, a horse, a pig, a rabbit, and a ferret. In some embodiments, the subject is a human. The terms “subject,” “individual,” and “patient” are used interchangeably, and as used herein are intended to include human and non-human animals. Non-human animals include all vertebrates, for example, mammals and non-mammals, such as non-human primates, sheep, dogs, rats, cats, cows, horses, ferrets, chickens, amphibians, and reptiles. Examples of mammals include non-human primates, sheep, dogs, cats, cows, ferrets, and horses.

### Further Embodiments

Provided herein is embodiment A, a method of treating a cancer or a neurological disease, the method comprising administering to a subject in need thereof an effective amount of quercetin, vitamin B3, vitamin C, and zafirlukast.

In an embodiment B, the method of embodiment A, wherein the cancer is selected from any of the group comprising ovarian cancer, lung cancer, prostate cancer, pancreatic cancer, colorectal cancer, endometrial cancer, brain/CNS cancer, liver cancer, thyroid cancer, bladder cancer, breast cancer, kidney cancer, cervical cancer, renal cancer, lymphoma, leukemia, skin cancer, urothelial cancer, glioblastoma, colorectal cancer, acute lymphocytic leukemia, acute myeloid leukemia, adrenal cancer, adult soft tissue sarcoma, anal cancer, aplastic anemia, basal and squamous cell skin cancer, bile duct cancer, bladder cancer, bone cancer, Castleman's disease, chronic lymphocytic leukemia, chronic myeloid leukemia, chronic myelomonocytic leukemia, endometrial cancer, esophageal cancer, Ewing family of tumors, eye cancer, gallbladder cancer, gastric cancer, gastrointestinal carcinoid, gastrointestinal stromal tumors, gestational trophoblastic disease, Hodgkin's disease, Kaposi's sarcoma, laryngeal and hypopharyngeal cancer, pediatric leukemia, non small cell lung cancer, small cell lung cancer, lung carcinoid tumor, malignant mesothelioma, melanoma skin cancer, multiple myeloma, myelodysplastic syndrome, nasal cavity and paranasal sinus cancer, nasopharyngeal cancer, neuroblastoma, non-Hodgkin's lymphoma, oral cavity and oropharyngeal cancer, osteosarcoma, penile cancer, pituitary tumors, renal cell carcinoma, retinoblastoma, rhabdomyosarcoma, salivary gland cancer, skin lymphoma, small intestine cancer, stomach cancer, testicular cancer, thymus cancer, thyroid cancer, uterine sarcoma, urothelial cancer, vaginal cancer, vulvar cancer, Waldenstrom's macroglobulinemia, and Wilms' tumor and combinations thereof.

In an embodiment C, the method of embodiment A, wherein the neurological disease is selected from any of the group comprising age-associated memory impairment (AAMI), age-related cognitive decline (ARCD), agitation synucleinopathies, Alzheimer's disease (AD), amyotrophic lateral sclerosis (ALS) dementia, autosomal-dominant Parkinson's disease, chemotherapy-induced neuropathy (CIPN), cognitive impairment no dementia (CIND), dementia, Creutzfeldt-Jakob disease (CJD), diffuse Lewy body disease (DLBD) also known as dementia with Lewy bodies (DLB), disorders or conditions characterized by the presence of Lewy bodies, Down syndrome, dyskinesia, epilepsy, frontotemporal dementia (FTD), HIV-associated neurocognitive disorder (HAND), HIV dementia, Huntington's disease, incidental LBD, inherited LBD, Lewy body dysphagia, mild cognitive impairment (MCI), multiple sclerosis, multiple system atrophy (MSA), neuropathies (including but not limited to peripheral neuropathy, diabetic neuropathy and retinal neuropathy), olivopontocerebellar atrophy, Parkinson's disease (PD), preclinical Alzheimer's disease (PCAD), psychiatric disorders (including but not limited to schizophrenia, bipolar disorders, depression, mania, anxiety disorders, post-traumatic stress disorders, delirium, eating disorders, autism, REM sleep behavior disorder, hallucinations, attention-deficit hyperactivity disorders, and psychosis), pure autonomic failure, seizures, Shy-Drager syndrome, striatonigral degeneration, synucleinopathies, traumatic brain injury (TBI), combined Alzheimer's and Parkinson disease and/or MSA, vascular dementia, diseases, disorders or conditions associated with abnormal expression, stability, activities and/or cellular processing of α-synuclein, diseases, disorders or conditions characterized by the presence of Lewy bodies, and combinations thereof.

In an embodiment D, the method of embodiment A, wherein the effective amount of quercetin is from about 500 mg to about 3000 mg.

In an embodiment E, the method of embodiment A, wherein the effective amount of vitamin B3 is from about 20 μg to about 3 g.

In an embodiment F, the method of embodiment A, wherein the effective amount of vitamin C is from about 200 μg to about 3 g.

In an embodiment G, the method of an embodiment A, further comprising administering an effective amount of folic acid.

In an embodiment H, the method of an embodiment G, wherein the effective amount of folic acid is from about 1000 μg to about 3000 μg.

In an embodiment I, the method of embodiment A, wherein the effective amount of zafirlukast is from about 10 mg to about 240 mg.

In an embodiment J, the method of embodiment A, wherein the effective amount is sufficient to halt progression of one or more symptoms of a cancer.

In an embodiment K, the method of embodiment A, wherein the effective amount is sufficient to halt progression of one or more symptoms of a neurological disease.

Provided herein is embodiment L, a method of treating a cancer or a neurological disease, the method comprising administering to a subject in need thereof an effective amount of quercetin, vitamin B3, vitamin C, and folic acid.

In an embodiment M, the method of embodiment L, wherein the effective amount of quercetin is from about 500 mg to about 3000 mg.

Provided herein is embodiment N, a method of treating a cancer or a neurological disease, the method comprising administering to a subject in need thereof an effective amount of zafirlukast.

In an embodiment O, the method of embodiment N, wherein the effective amount of zafirlukast is from about 10 mg to about 240 mg.

Provided herein is embodiment P, a method of treating a cancer or a neurological disease, the method comprising administering to a subject in need thereof an effective amount of zafirlukast and a chemotherapeutic agent.

In an embodiment Q, the method of embodiment P, wherein the effective amount of zafirlukast is from about 10 mg to about 240 mg of zafirlukast.

Provided herein is embodiment R, a method of treating a cancer or a neurological disease, the method comprising administering to a subject in need thereof an effective amount of quercetin, vitamin B3, vitamin C, a chemotherapeutic agent, and zafirlukast.

In an embodiment S, the method of embodiment R, further comprising administering an effective amount of folic acid.

### EXAMPLES

### Example 1

### Treating ALS with IsoQuercetin

Background: Underlying pathology and mechanism for ALS seems to revolve around four key activities: mast cell aberrations and over expression, iron metabolism and ferritin levels, PDI and thiol isomerase activity, and microglial activation.

New evidence suggests mast cell autocrine signaling contributes to ALS development. Mast cells are known to disrupt the permeability of the blood-brain barrier. The mast cell chemoattractant interleukin 15 (IL15) is elevated in the serum and cerebralspinal fluid (CSF) of ALS patients and interleukin 17 (IL17) has been found in the spinal cord of ALS patients. Additional evidence that mast cell signaling contributes to ALS development is seen in SOD193A rats, where KIT proto-oncogene receptor tyrosine kinase (c-KIT) expression in mast cells mediates inflammation and neuromuscular denervation. Mast cells are abundant in ALS patients, where degranulating mast cells have been found in quadriceps of ALS patients while patients without ALS did not have evidence of any degranulating mast cells.

Iron metabolism is dysregulated in ALS patients and linked with ALS pathogenesis (Wang et al., 2020; which is incorporated by reference herein in its entirety). Evidence suggests that iron accumulation is involved in onset and progression of ALS (Xian-Le-Bu et al 2019; which is incorporated by reference herein in its entirety) and ferritin levels closely correlate with disease duration.

Endoplasmic reticulum (ER)-resident protein-disulfide isomerase (PDI) family members are significantly upregulated in lumbar spinal cords of the ALS rat model SOD1G93A. Neurotoxicity results from PDI activity, which activates ERO1α, which in turn produces hydrogen peroxide. Additionally, overexpression of PDI from endothelial cells results in mast cell overexpression and results in endothelial cell wall being breached.

Microglial activation is a hallmark of ALS. Microglia proliferation and activation results in release of a variety of cytokines can have either protective or detrimental effects on neurons (Li and Barres, 2018; which is incorporated by reference herein its entirety).

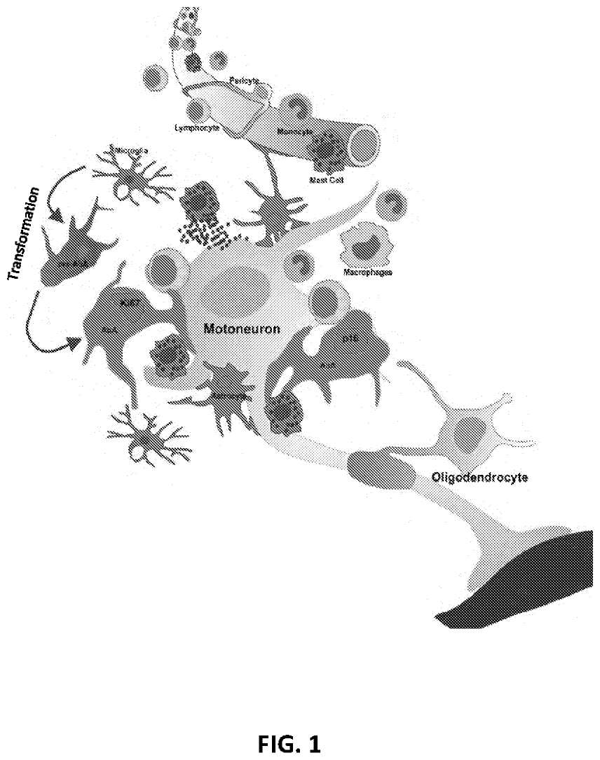

In summary, the cellular microenvironment contributes to the degeneration in ALS. IsoQ can be used to modulate the microenvironment and treat, prevent or delay the onset of ALS. IsoQ downregulates neurotoxic microglia and senescent microglia and acts as a mast cell expression modulator, blocking extracellular PDI and associated inflammatory and neurotoxic pathways. IsoQ downregulates mast cells that infiltrate along the motor pathways. IsoQ targets mitochondria pathology in motor axons and myofibers and acts as a ferroportin inhibitor (FIG. 1).

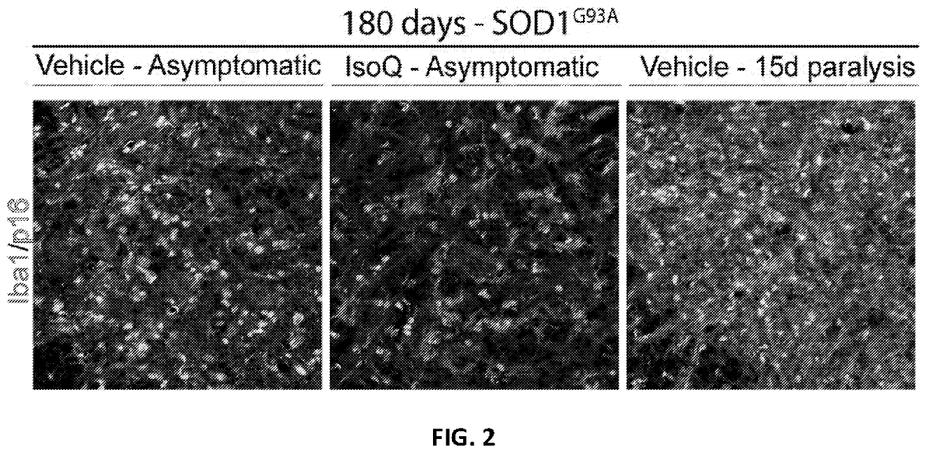

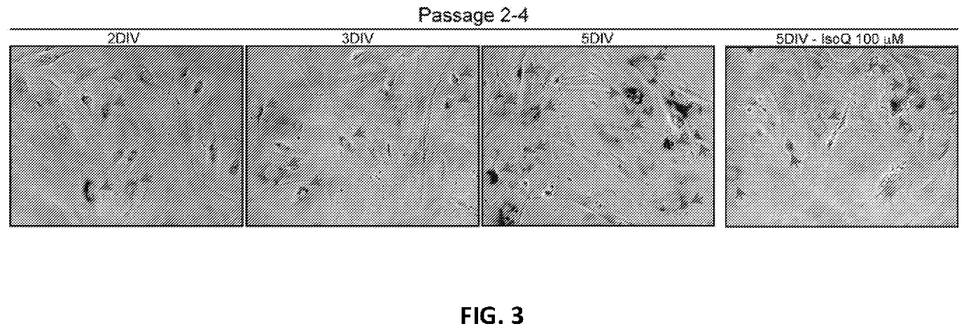

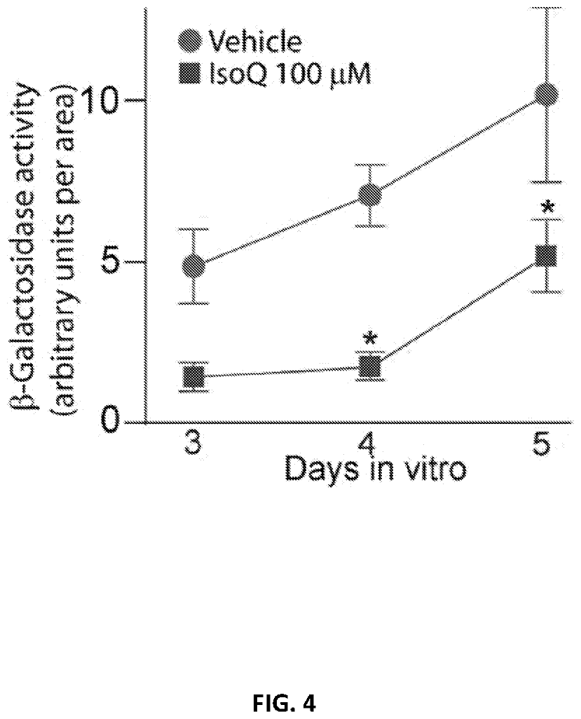

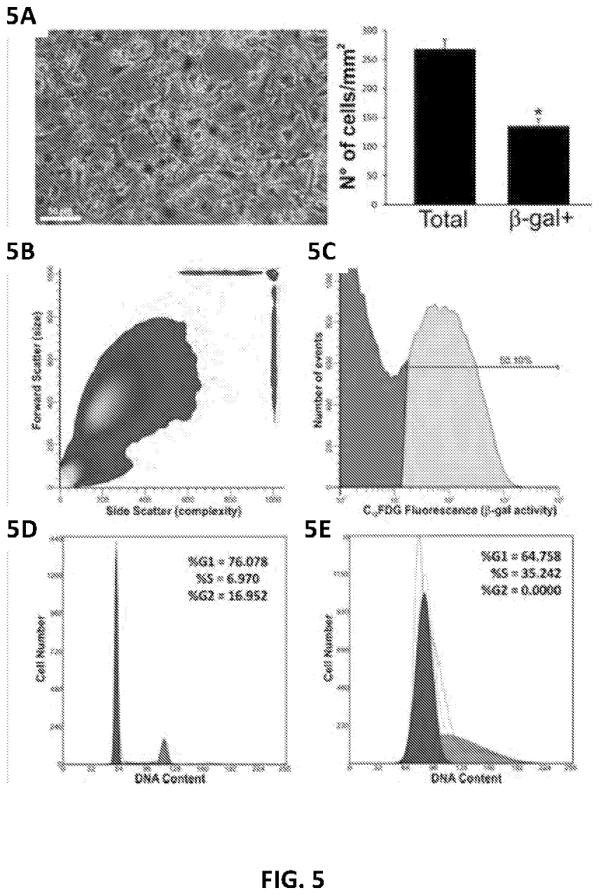

Experiments and Results: Treatment of SOD1G93A rats, a rat model of ALS, with IsoQ potently downregulated senescent microglia number in the spinal cord (FIG. 2; Ibal =microglia; p16 =senescence marker). B-Gal activity demonstrated that IsoQ downregulated senescence markers in cultured ALS microglia (FIG. 3; FIG. 4; FIG. 5; 12 hours IsoQ treatment).

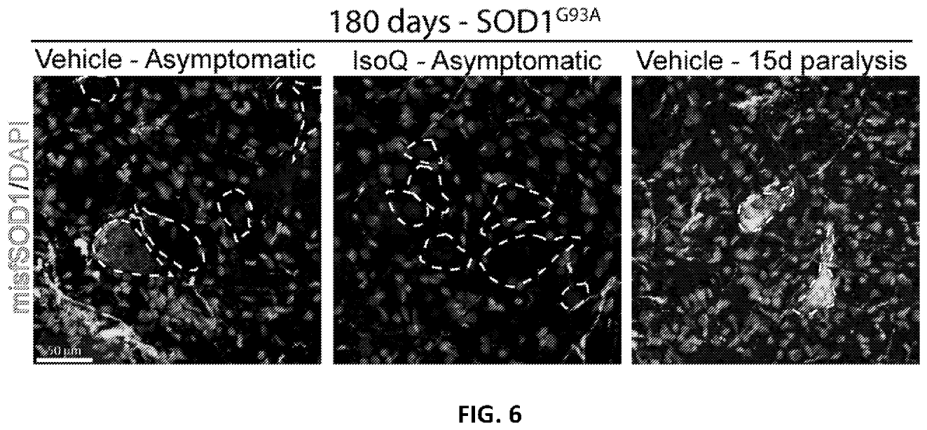

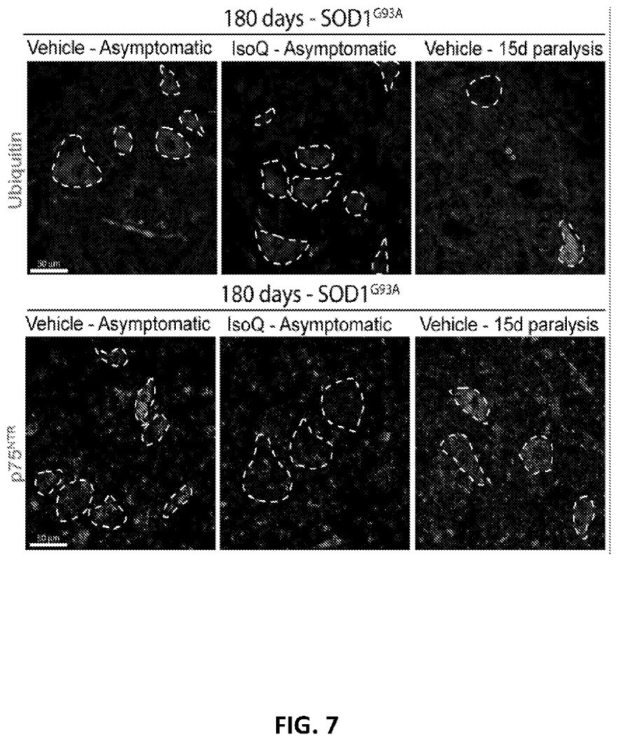

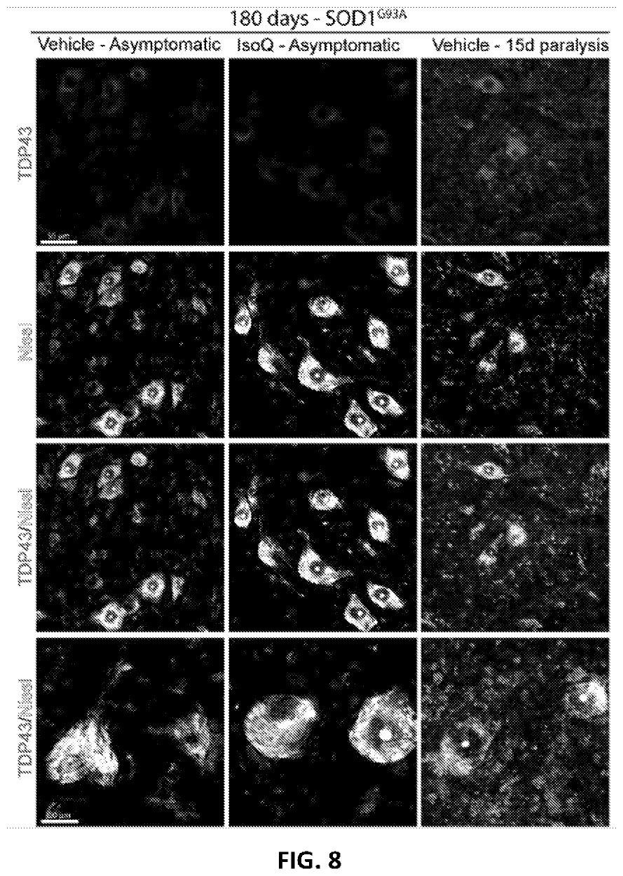

IsoQ treatment in presymptomatic SOD1G93A rats preserved the number of healthy motor neurons compared to asymptomatic controls, with an apparent net effect on misfolding of SOD 1 as well as p75NTR and TDP43 upregulation. Spinal motor neuron pathology and SOD1 misfolding improved after treatment with IsoQ (FIG. 6). Further, IsoQ prevented ubiquitin and p75NTR expression in damaged motor neurons (FIG. 7). TDP-43 aggregates in motor neurons were prevented with IsoQ treatment (FIG. 8).

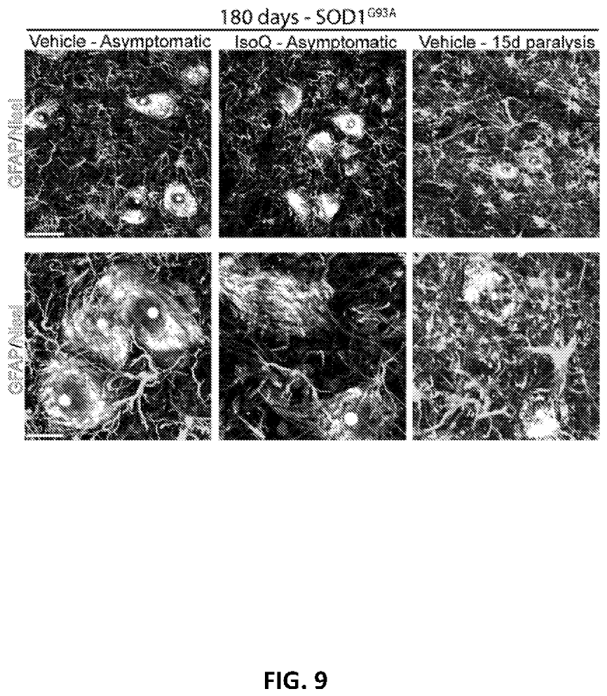

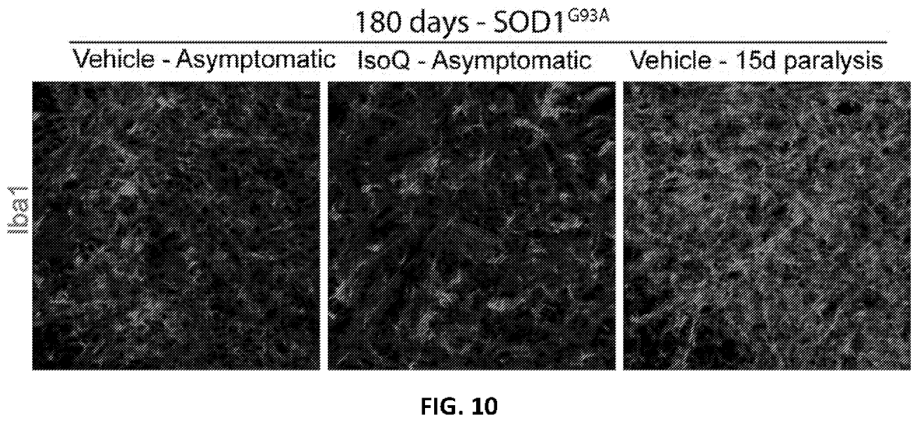

Treatment of the spinal cord ventral horn of SOD1G93A rats decreased astrocytosis in the ventral horn (FIG. 9) as well as microgliosis (FIG. 10).

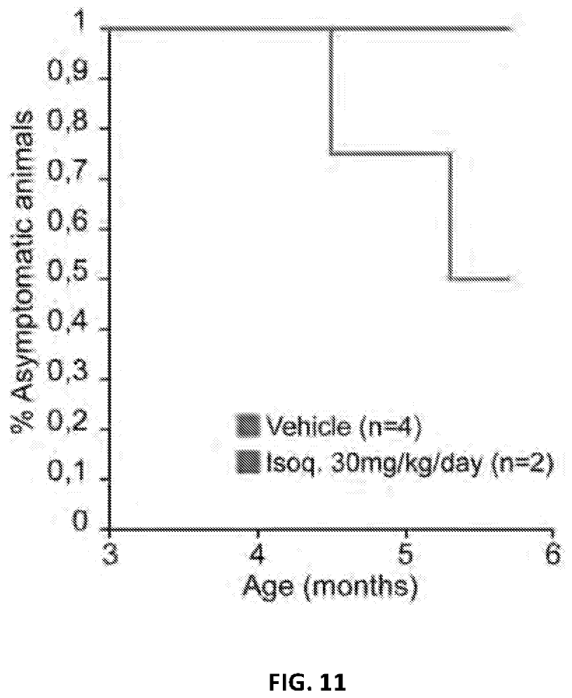

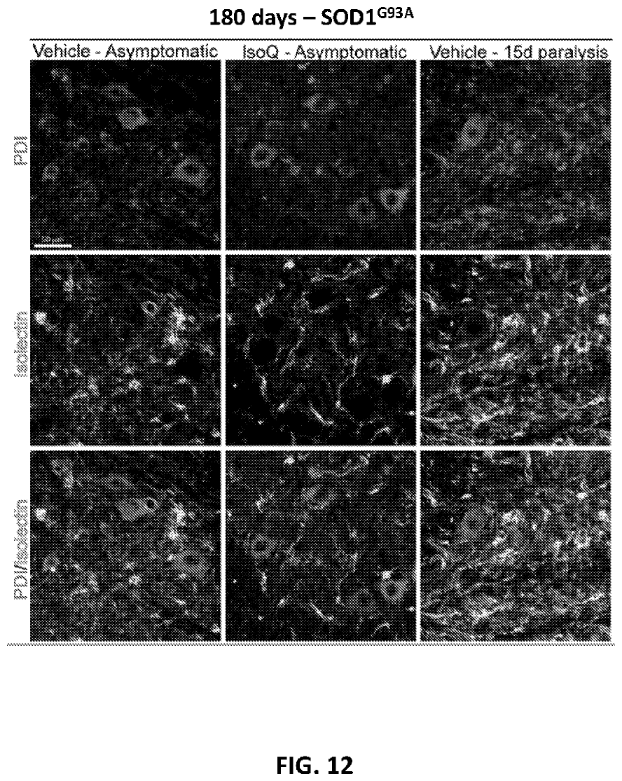

Treatment with IsoQ prevented development of symptoms in SOD1G93A rats over a three-month experimental window (FIG. 11). Animals (male littermates) were administered 30 mg/kg of IsoQ (95%)-vit C (5%)-niacin/vitamin B3 (1%) in water daily. PDI was upregulated in motor neurons of SOD1G93A rats, while animals treated with IsoQ had similar levels of PDI expression compared to asymptomic mice (FIG. 12). PDI expression in the sciatic nerve and spinal cord was low in non-symptomatic rats but sharply increased after paralysis onset. These results show for the first time that in the spinal cord of paralytic rats, PDI is mainly localized extracellularly, suggesting an active secretion of the protein. PDI may also be secreted from motor neurons and astrocytes. PDI secretion from glial cells (astrocytes and Schwann cells) could be a mechanism to elicit immune cell infiltration in ALS (neutrophils), which has a recognized pathogenic role in mediating distal axon neuropathy. Notably, treatment with IsoQ prior to disease onset prevented extracellular PDI upregulation in the spinal cord, suggesting a mechanism of neuroprotection.

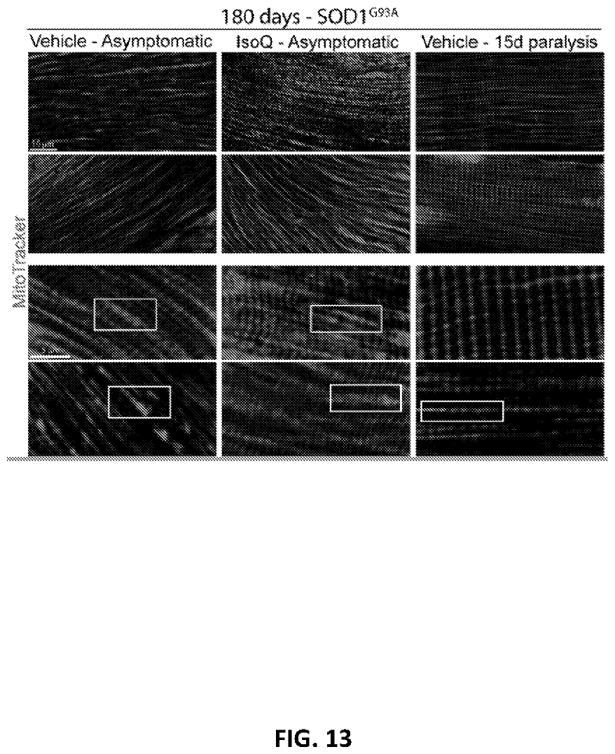

To understand whether IsoQ modulates muscle mitochondria, myofibers from extensor digitorum longus (EDL) muscle were obtained from SOD1G93A rats prior to perfusion with paraformaldehyde. IsoQ treatment in pre-symptomatic SODIG93A rats preserved mitochondria number, integrity and size, and increased ‘long mitochondria domains’ characteristic of healthy oxidative myofibers (FIG. 13).

Treatment with IsoQ prior to symptom delayed the development of neuronal and glial molecular markers that precede paralysis onset. IsoQ also ameliorated mitochondria status in skeletal muscle. These results suggest that the most relevant therapeutic targets are: extracellular accumulation of PDI; mitochondria number and size; motor neuron cell body pathology; accumulation of senescent microglia; mast cell infiltration; ferroportin inhibition and iron deregulation of iron metabolism.

The combination of IsoQ and zafirlukast reaches many aspects of ALS pathology: inflammation, mast cells, ferroportin inhibition, and protection of microglia. As such, the combination of zafirlukast and IsoQ present a unique opportunity to prevent, delay or treat symptoms of ALS.

What is claimed is:

1. A method of treating a cancer or a neurological disease, the method comprising administering to a subject in need thereof an effective amount of quercetin, vitamin B3, vitamin C, and zafirlukast.

2. The method of claim 1, wherein the cancer is selected from any of the group comprising ovarian cancer, lung cancer, prostate cancer, pancreatic cancer, colorectal cancer, endometrial cancer, brain/CNS cancer, liver cancer, thyroid cancer, bladder cancer, breast cancer, kidney cancer, cervical cancer, renal cancer, lymphoma, leukemia, skin cancer, urothelial cancer, glioblastoma, colorectal cancer, acute lymphocytic leukemia, acute myeloid leukemia, adrenal cancer, adult soft tissue sarcoma, anal cancer, aplastic anemia, basal and squamous cell skin cancer, bile duct cancer, bladder cancer, bone cancer, Castleman's disease, chronic lymphocytic leukemia, chronic myeloid leukemia, chronic myelomonocytic leukemia, endometrial cancer, esophageal cancer, Ewing family of tumors, eye cancer, gallbladder cancer, gastric cancer, gastrointestinal carcinoid, gastrointestinal stromal tumors, gestational trophoblastic disease, Hodgkin's disease, Kaposi's sarcoma, laryngeal and hypopharyngeal cancer, pediatric leukemia, non small cell lung cancer, small cell lung cancer, lung carcinoid tumor, malignant mesothelioma, melanoma skin cancer, multiple myeloma, myelodysplastic syndrome, nasal cavity and paranasal sinus cancer, nasopharyngeal cancer, neuroblastoma, non-Hodgkin's lymphoma, oral cavity and oropharyngeal cancer, osteosarcoma, penile cancer, pituitary tumors, renal cell carcinoma, retinoblastoma, rhabdomyosarcoma, salivary gland cancer, skin lymphoma, small intestine cancer, stomach cancer, testicular cancer, thymus cancer, thyroid cancer, uterine sarcoma, urothelial cancer, vaginal cancer, vulvar cancer, Waldenstrom's macroglobulinemia, and Wilms' tumor and combinations thereof.

3. The method of claim 1, wherein the neurological disease is selected from any of the group comprising age-associated memory impairment (AAMI), age-related cognitive decline (ARCD), agitation synucleinopathies, Alzheimer's disease (AD), amyotrophic lateral sclerosis (ALS) dementia, autosomal-dominant Parkinson's disease, chemotherapy-induced neuropathy (CIPN), cognitive impairment no dementia (CIND), dementia, Creutzfeldt-Jakob disease (CJD), diffuse Lewy body disease (DLBD) also known as dementia with Lewy bodies (DLB), disorders or conditions characterized by the presence of Lewy bodies, Down syndrome, dyskinesia, epilepsy, frontotemporal dementia (FTD), HIV-associated neurocognitive disorder (HAND), HIV dementia, Huntington's disease, incidental LBD, inherited LBD, Lewy body dysphagia, mild cognitive impairment (MCI), multiple sclerosis, multiple system atrophy (MSA), neuropathies (including but not limited to peripheral neuropathy, diabetic neuropathy and retinal neuropathy), olivopontocerebellar atrophy, Parkinson's disease (PD), preclinical Alzheimer's disease (PCAD), psychiatric disorders (including but not limited to schizophrenia, bipolar disorders, depression, mania, anxiety disorders, post-traumatic stress disorders, delirium, eating disorders, autism, REM sleep behavior disorder, hallucinations, attention-deficit hyperactivity disorders, and psychosis), pure autonomic failure, seizures, Shy-Drager syndrome, striatonigral degeneration, synucleinopathies, traumatic brain injury (TBI), combined Alzheimer's and Parkinson disease and/or MSA, vascular dementia, diseases, disorders or conditions associated with abnormal expression, stability, activities and/or cellular processing of α-synuclein, diseases, disorders or conditions characterized by the presence of Lewy bodies, and combinations thereof.

4. The method of claim 1, wherein the effective amount of quercetin is from about 500 mg to about 3000 mg.

5. The method of claim 1, wherein the effective amount of vitamin B3 is from about 20 μg to about 3 g.

6. The method of claim 1, wherein the effective amount of vitamin C is from about 200 μg to about 3 g.

7. The method of claim 1, further comprising administering an effective amount of folic acid.

8. The method of claim 7, wherein the effective amount of folic acid is from about 1000 μg to about 3000 μg.

9. The method of claim 1, wherein the effective amount of zafirlukast is from about 10 mg to about 240 mg.

10. The method of claim 1, wherein the effective amount is sufficient to halt progression of one or more symptoms of a cancer.

11. The method of claim 1, wherein the effective amount is sufficient to halt progression of one or more symptoms of a neurological disease.

12. A method of treating a cancer or a neurological disease, the method comprising administering to a subject in need thereof an effective amount of quercetin, vitamin B3, vitamin C, and folic acid.

13. The method of claim 12, wherein the effective amount of quercetin is from about 500 mg to about 3000 mg.

14. A method of treating a cancer or a neurological disease, the method comprising administering to a subject in need thereof an effective amount of zafirlukast.

15. The method of claim 14, wherein the effective amount of zafirlukast is from about 10 mg to about 240 mg.

16. A method of treating a cancer or a neurological disease, the method comprising administering to a subject in need thereof an effective amount of zafirlukast and a chemotherapeutic agent.

17. The method of claim 16, wherein the effective amount of zafirlukast is from about 10 mg to about 240 mg of zafirlukast.

18. A method of treating a cancer or a neurological disease, the method comprising administering to a subject in need thereof an effective amount of quercetin, vitamin B3, vitamin C, a chemotherapeutic agent, and zafirlukast.

19. The method of claim 18, further comprising administering an effective amount of folic acid.
